(19) United States
(12) Patent Application Publication
(10) Pub. No.: US 2025/0049666 A1
(43) Pub. Date: Feb. 13, 2025

(54) LIQUID COMPOSITION HAVING ENHANCED ANTIMICROBIAL ACTIVITY

(71) Applicant: Conopco Inc., d/b/a UNILEVER, Englewood Cliffs, NJ (US)

(72) Inventors: Prem Chandar, Closter, NJ (US); Tirucherai Varahan VASUDEVAN, Bethany, CT (US); Nicholas Arthur VELEZIS, Naugatuck, CT (US); Connor Patrick WALSH, Seymour, CT (US)

(73) Assignee: Conopco Inc., d/b/a UNILEVER, Englewood Cliffs, NJ (US)

(21) Appl. No.: 18/723,765

(22) PCT Filed: Dec. 19, 2022
(86) PCT No.: PCT/EP2022/086601

§ 371 Date: Jun. 24, 2024

(30) Foreign Application Priority Data
Dec. 27, 2021 (EP) 21217787.7

(51) Int. Cl. A61K 8/368; A61K 8/46; A61Q 5/02; A61Q 19/10

(52) U.S. Cl. CPC A61K 8/368; A61K 8/466; A61Q 5/02; A61Q 19/10; A61K 2800/30; A61K 2800/596

(57) ABSTRACT

The invention is directed to a mild liquid composition with enhanced antimicrobial activity. The liquid composition of the present invention is mild and unexpectedly yields superior antimicrobial activity when used in cleansing applications. The liquid composition comprises an anionic surfactant and an acid, and is formulated to yield a wash solution having a pH from 2.9 to 5.2.

### FIELD OF THE INVENTION

The present invention is directed to a mild liquid composition with enhanced antimicrobial activity. More particularly, the liquid composition of the present invention is mild and unexpectedly yields superior antimicrobial activity when used in cleansing applications. The liquid composition comprises an anionic surfactant and an acid, and is formulated to yield a wash solution having a pH from 2.9 to 5.2. Such a liquid composition may be made via conventional mixing techniques, and can be formulated substantially free of or free of ethanol, parabens, formaldehyde donors, silicones, sulfates, glycinates and/or halogenated antimicrobial actives.

### BACKGROUND OF THE INVENTION

Cleansing compositions, including bars and liquids, are known to provide many benefits to consumers using them. Germs, in general, can accumulate on hands, hair, cloths and hard surfaces and they may be passed from person to person or object to person via the air and/or routine and ordinary contact. Once on the hands, for example, infection from germs is inevitable since consumers are constantly touching their eyes, nose and mouth, resulting in a host of illnesses, including respiratory and gastrointestinal illnesses. The Center for Disease Control reports that washing hands with soap removes germs much more effectively than water alone and reduces the chances of getting common aliments like diarrhea and the common cold. In fact, regular use of cleansing compositions reduces the amount of antibiotics people use and the likelihood that antibiotics will become less effective to those in need of them.

Additionally, cleansing compositions traditionally have biocidal action against many Gram negative bacteria. The biocidal action of wash compositions against Gram negative and Gram positive bacteria is considerably much less common, and especially, when the compositions are substantially free of soap, sulfates and halogenated actives, and formulated to be very mild on skin.

There is an increasing interest to develop a liquid composition that is mild and yields exceptional antimicrobial activity when used on skin, hair, clothing, hard surfaces or the like. This invention, therefore, is directed to a liquid composition that unexpectedly yields superior antimicrobial activity when used in cleansing applications. The liquid composition comprises an anionic surfactant and an acid, and is formulated to generate or yield a wash solution having a pH from 2.9 to 5.2. Such compositions may be made via conventional mixing techniques, and they can be formulated substantially free of or free of ethanol, parabens, formaldehyde donors, silicones, sulfates, glycinates and/or halogenated antimicrobial actives.

### ADDITIONAL INFORMATION

Efforts have been disclosed for making antibacterial soap bars. In U.S. Pat. No. 6,794,344, soap based bar compositions having 50% soap with an alkyl chain length of 8 to 10 carbon atoms are described.

Other efforts have been disclosed for making soap-based formulations. In U.S. Patent Application Publication No. 2010/0098776, disclosed are soap-based liquid wash formulations with enhanced deposition of conditioning and/or skin appearance enhancing agents.

Even other efforts have been disclosed for making soap bar compositions. In WO16024090A1, a semi-translucent soap bar composition with antimicrobial benefit is described.

Still other efforts have been disclosed for making antimicrobial formulations. In U.S. Pat. No. 11,110,048, topical sanitizing formulations with quaternary ammonium salts are described.

None of the additional information describes a liquid composition having enhanced antimicrobial activity as claimed herein.

### SUMMARY OF THE INVENTION

In a first aspect, the present invention is directed to liquid composition comprising:
a) from 2 to 20% by weight anionic surfactant;
b) from 0.2 to 8% by weight of at least one acid having a pKa from 1 to 8 and a log P from 0 to 4.95;
c) water; and
d) optionally, a liquid structuring agent,
   wherein the composition in a 50% aqueous solution has a pH from 2.9 to 5.2 and further wherein the composition is substantially sulfate free.

In a second aspect, the invention is directed to method for reducing antimicrobial presence (both Gram negative and Gram positive) on a surface in need of antimicrobial reduction comprising the steps of:
a) contacting the surface with a wash solution comprising liquid composition from the first aspect of the invention and water; and
b) rinsing the wash solution from the surface,
wherein the liquid composition has an antimicrobial activity in a 50% wash solution that provides a Log₁₀ Reduction against E. coli ATCC 10536 and S. aureus ATCC 6538 of at least 1.0 after 15 seconds (preferably after 10 seconds) of contact, and preferably, of at least 2.0 after 25 seconds (preferably after 20 seconds) of contact, and most preferably, of at least 2.75 (preferably 3.0) after 40 seconds (preferably after 35 seconds, and most preferably, after 30 seconds) of contact with a surface (e.g., skin or a counter top) in need of antimicrobial reduction.

In a third aspect, the present invention is directed to a use of the liquid composition of the first aspect of the invention to provide in a 50% aqueous solution a Log₁₀ reduction against E. coli ATCC 10536 and S. aureus ATCC 6538 of at least 1.0 after 15 seconds of contact, and preferably, of at least 2.0 after 25 seconds of contact, and most preferably, of at least 3.0 after 35 seconds of contact with a surface (e.g., skin or a counter top) in need of antimicrobial reduction.

Skin, as used herein, is meant to include skin on the arms (including underarms), face, feet, neck, chest, hands, legs, buttocks and scalp (including hair). Liquid composition (i.e., an end use composition) is defined to mean a composition ready to use in a cleansing application, and therefore, the same can be a shampoo, make-up remover, face cleanser or personal wash for body and/or hands. Such a liquid composition is also suitable to use as a home care cleanser (for an inanimate object), like a cleanser for an automobile, table, window, sink, toilet or a cleanser for laundry (inanimate objects). In an embodiment of the invention, the liquid composition is a personal wash composition for skin. In another embodiment, the liquid composition is a personal wash composition for the hands. The liquid composition is typically used with 30 to 70% by weight composition to 70 to 30% by weight water to produce an aqueous solution or wash solution having a pH from 2.9 to 5.2. For the avoidance of doubt, a 50% aqueous (or wash) solution means 50% by weight of water and 50% by weight of the liquid composition or end use composition; and therefore, a 1:1 water to liquid composition dilution where the liquid composition itself would be formulated with water not inclusive of the water used for dilution. The pH of the liquid composition would be essentially the same as the wash solution. The liquid composition of the present invention is suitable to deliver in a wash solution an active pharmaceutical ingredient (e.g., like corticoids, antihistamines and/or antibiotics). In a preferred embodiment, the liquid composition and resulting wash solution are cosmetic and non-therapeutic compositions suitable to remove soil (water and fat soluble) from skin, and especially, skin on the hands. The liquid composition and wash solution is surprisingly mild while simultaneously delivering antimicrobial benefits. Mild, as used herein, means having a Zein value ≤7 as defined and determined via the method described in the Examples. Antimicrobial benefits means a 50% wash solution provides a Log₁₀ reduction against E. coli ATCC 10536 and S. aureus ATCC 6538 of at least 1.0 after 15 seconds of contact, and preferably, of at least 2.0 after 25 seconds of contact, and most preferably, at least 3 after 35 seconds of contact with a surface, like skin, in need of antimicrobial reduction. Liquid structuring agent means a component that contributes to the stability of the liquid composition as a lamellar composition. As used herein, pKa is the −log₁₀ Ka where Ka is the acid dissociation constant. Log P is the log₁₀ (Partition Coefficient) where the partition coefficient, P is equal to [Compound]_{octanol}/[Compound]_{water}.

Substantially free of means no more than 3.5% by weight of the total weight of the liquid composition, and preferably, less than 1.5% by weight of the total weight of liquid composition. In still another embodiment, substantially free of means less than 1%, and preferably, less than 0.5% by weight of the total weight of the liquid composition. Substantially free of, as used herein, is meant to include 0.00001% to 3.5%, or 0.00001 to 1%, or 0.00001 to 0.5%, or 0.0% by weight of the total weight of the liquid composition. As to soap, substantially free (of soap) means less than 5.5%, and preferably, less than 4.5%, and most preferably, less than 3.75% by weight soap based on total weight of the liquid composition. In an embodiment of the invention, the liquid composition comprises from 0.00001 to 3.5% or 0.001 to 3.35% by weight soap. In another embodiment, the liquid composition comprises no (0.0% by weight) soap. Betaine (or sultaine) as used herein is meant to mean a surfactant derived from the same. In the case of betaine, for example, a surfactant derived therefrom is cocoamidopropyl betaine.

In still another embodiment of the invention, the liquid composition can be formulated to be substantially free of ethanol, paraben, formaldehyde donors, sulfates, glycinate, glutamate, halogenated antimicrobial actives and any ingredients tested on animals. In yet another embodiment of the invention, the aqueous solution or wash solution (1:1, respectively, water to liquid composition) comprises less than 1% by weight or 0.0% by weight of C₁₆₋₁₈ methyl ester sulphonate, glycinate, carboxylate, glutamate and/or alpha olefin sulfonate. In still another embodiment, the wash composition of the same dilution comprises less than 2% by weight silicone or 0.0% by weight silicone (e.g., no cyclosiloxane like decamethylcyclopentasiloxane).

Skin benefit agents can optionally be used in the liquid composition of the present invention and such agents may be water or oil soluble. The liquid composition will have a viscosity from 2,000 to 15,000 mPa·s and the 50% aqueous solution will have a viscosity from 20 to 1,500 mPa·s where viscosity is taken with a Brookfield helipath TD, at 4 rpm for 1 minute at 25° C. unless noted otherwise. In the absence of explicitly stating otherwise, all ranges described herein are meant to include all ranges subsumed therein. The term comprising is meant to encompass the terms consisting essentially of and consisting of. For the avoidance of doubt, and for illustration, an aqueous solution comprising water, anionic surfactant and acid is meant to include a solution consisting essentially of the same and a solution consisting of the same. Except in the operating comparative examples, or where otherwise explicitly indicated, all numbers in this description indicating amounts or ratios of materials or conditions and/or physical properties of materials and/or use are to be understood as modified by the word “about”.

### DETAILED DESCRIPTION OF THE INVENTION

As to anionic surfactants suitable for use in the liquid composition of the present invention, such surfactants can include glutamates, aspartates, aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or an alkyl glyceryl ether sulfonate (AGSs) as well as aromatic sulfonates such as alkyl benzene sulfonate.

The anionic surfactant used in the present invention can optionally include some alkyl sulfate (e.g., C₁₂-C₁₈ alkyl sulfate) and/or alkyl ether sulfate (including alkyl glyceryl ether sulfates). As noted herein, the liquid composition of the present invention is substantially sulfate free (e.g., sulfate-based surfactant free). If used, the sulfates suitable include those having the formula:

RO(CH₂CH₂O)ₙSO₃M

wherein R is an alkyl or alkenyl having 8 to 18 carbons, preferably 12 to 18 carbons, n has an average value of at least 1.0, preferably less than 5, and most preferably 1 to 4, and M is a solubilizing cation such as sodium, potassium, ammonium or substituted ammonium. As noted, sulfates, when optionally used, make 3.5% or less by weight of the liquid composition.

In an embodiment of the invention, the anionic may also include alkyl sulfosuccinates (including mono- and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:

R¹O₂CCH₂CH(SO₃M)CO₂M;

and amide-MEA sulfosuccinates of the formula:

R¹CONHCH₂CH₂O₂CCH₂CH(SO₃M)CO₂M

wherein R¹ is a C₈-C₂₂ alkyl group.

Sarcosinates suitable for use are generally represented by the formula:

R²CON(CH₃)CH₂CO₂M,

wherein R² is a C₈-C₂₀ alkyl group.

Taurates suitable for use are generally identified by formula:

R³CONR⁴CH₂CH₂SO₃M

wherein R³ is a C₈-C₂₀ alkyl group, R⁴ is a C₁-C₄ alkyl group, and M is a solubilizing cation as previously described.

Isethionates suitable for use in the liquid composition may include C₈-C₁₈ acyl isethionates (including those which have a substituted head group such as a C₁₋₄ alkyl substitution, preferably methyl substitution). These esters are typically prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms. Isethionates having at head substituted isomers are described in world application WO 94/09763 and U.S. Pat. Nos. 2,810,747 and 2,820,818, the disclosures of which are incorporated herein by reference.

The acyl isethionate that may be used can be an alkoxylated isethionate such as those described in Ilardi et al., U.S. Pat. No. 5,393,466, entitled “Fatty Acid Esters of Polyalkoxylated isethonic acid; issued Feb. 28, 1995; hereby incorporated by reference. This compound has the general formula:

R⁵C—O(O)—C(X)H—C(Y)H—(OCH₂—CH₂)ₘ—SO₃M

wherein R⁵ is an alkyl group having 8 to 18 carbons, m is an integer from 1 to 4, X and Y are each independently hydrogen or an alkyl group having 1 to 4 carbons and M is a solubilizing cation as previously described.

Illustrative examples of glutamates suitable for use in the present invention include sodium lauroyl glutamate, sodium cocoyl glutamate, potassium lauroyl glutamate, potassium cocoyl glutamate, disodium cocoyl glutamate, mixtures thereof or the like. Illustrative examples of aspartates suitable for use either alone or together with glutamates and/or other anionic surfactants include sodium lauroyl aspartate, potassium lauroyl aspartate, sodium cocoyl aspartate, potassium cocoyl aspartate, mixtures thereof or the like.

Citrates, including laureth-7 citrate, are suitable for use as anionic surfactants as are carboxylates like sodium laureth-5 carboxylate, sodium lauryl glucose carboxylate, sodium alkyl pareth-8 carboxylates and sodium alkyl pareth-12 carboxylates. Sulfonated tallow fatty acids are yet another class of anionics suitable for use.

Succinates suitable for use in the liquid composition of the present invention include disodium laureth sulfosuccinate, disodium lauryl sulfosuccinate, MEA-sulfosuccinate or mixtures thereof. Sarcosinates like sodium and/or potassium lauroyl sarcosinate is/are also suitable for use.

In one embodiment of the invention, the anionic surfactant used is sodium cocoyl isethionate, sodium lauroyl isethionate, sodium myristoyl isethionate, sodium oleoyl isethionate, ammonium oleoyl isethionate or a mixture thereof. Other isethionates suitable for use include acyl alkyl isethionate esters including acyl 1-alkyl, 2-alkyl and 1,2-alkyl isethionates where the alkyl group is often a C₁-C₄-alkyl, and preferably, a C₁ and/or C₂ alkyl, and most preferably, a methyl group. In an embodiment of the invention, a mixture of acyl 1-alkyl and acyl 2-alkyl isethionates in a weight ratio from 1:25 to 1:2, and preferably, 1:20 to 1:3, and most preferably, from 1:18 to 1:4, respectively, may be used where the alkyl group is a C₁-C₆ alkyl and preferably a methyl and/or ethyl group. In still another embodiment of the invention, the acyl alkyl isethionate is sodium lauroyl methyl isethionate in a mixture where at least 50 to 95%, and preferably, 60 to 85%, and most preferably, 65 to 80% by weight of the methyl group is on carbon two of the isethionate group, based on total weight of the isethionate in the liquid composition.

In still another embodiment, the anionic surfactant is a taurate like sodium lauroyl taurate, sodium methyl lauroyl taurate, sodium methyl cocoyl taurate, sodium methyl oleoyl taurate, sodium methyl myristoyl taurate, potassium methyl myristoyl taurate, calcium methyl lauroyl taurate, potassium methyl lauroyl taurate, ammonium methyl lauroyl taurate or a mixture thereof.

In yet another embodiment of the invention, the anionic surfactant used in the liquid composition of the present invention is a mixture of any of those described herein.

In, however, another embodiment, the anionic surfactant used is an isethionate, a taurate or a mixture thereof. When a mixture of isethionate and taurate, the mixture is typically in a taurate to isethionate weight ratio from 99:1 to 1:99, and preferably, from 15:85 to 85:15, and most preferably, from 25:75 to 75:25 based on total weight of taurate and isethionate in the liquid composition. Still in another embodiment, the weight ratio of taurate to isethionate in the liquid composition is 35:65 to 65:35 or 58:42 to 42:58 based on total weight of taurate and isethionate in the liquid composition.

Often, and in another embodiment, the anionic surfactant in the liquid composition of the present invention is at least 60% by weight isethionate and/or taurate, and preferably, at least 65% by weight isethionate and/or taurate, and most preferably, at least 70 to 100% by weight isethionate and/or taurate based on total weight of the anionic surfactant in the liquid composition. A liquid composition with anionic surfactant at 100% by weight isethionate or 100% by weight taurate (based on total weight of anionic surfactant in the liquid composition) is also within the scope of the present invention. It is also within the scope of the present invention for the liquid composition to comprise only anionic surfactant (i.e., 100%) as the surfactant in the composition, and therefore, a liquid composition with anionic surfactant and 0.0% by weight betaine is within the scope of the invention. In still another embodiment of the invention, anionic surfactant and zwitterionic surfactant are used at a weight ratio of anionic to zwitterionic surfactant of 5:1 to 1.35:1 or from 4:1 to 3.5:1 or from 3.5:1 to 2.75:1 or from 3:1 to 2.65:1. In even another embodiment of the invention, the anionic surfactant is a taurate, isethionate or both and the zwitterionic surfactant is a betaine. In an often desired embodiment, the anionic surfactant is a taurate and the zwitterionic surfactant is a betaine employed in the liquid composition at the defined weight ratios.

Anionic surfactant typically makes up from 2 to 20%, and preferably, from 3 to 15%, and most preferably, from 4 to 9% by weight of the liquid composition. In an embodiment of the invention, anionic makes up from 3 to 12% or 3 to 10% or from 4 to 9% by weight of the liquid composition.

When optionally desired, amphoteric surfactants suitable for use can be used in the invention (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈alkyl portion. Illustrative examples of the amphoteric surfactants suitable for use include sodium lauroamphoacetate, sodium cocoamphoacetate or mixtures thereof. If present such surfactants make up from 0.00001 to 5.0%, and preferably, from 0.1 to 4.5%, and most preferably, from 0.1 to 3.5% by weight of the liquid composition. The liquid composition can comprise 0.00001 to 2% by weight amphoteric surfactant and no (0.0%) by weight of the liquid composition.

As to zwitterionic surfactants that may optionally be employed in the present invention, such surfactants include at least one acid group. Such an acid group may be a carboxylic or a sulphonic acid group. They often include quaternary nitrogen, and therefore, can be quaternary amino acids. They often generally include an alkyl or alkenyl group of 7 to 18 carbon atoms and generally comply with an overall structural formula:

R⁶—[—C(O)—NH(CH₂)_{q}—]ᵣ—N⁺—(R⁷—)(R⁸)A-B

where R⁶ is alkyl or alkenyl of 7 to 18 carbon atoms; R⁷ and R⁸ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Suitable zwitterionic surfactants for optional use in the present invention include betaines of the formula:

R⁶—N⁺—(R⁷)(R⁸)CH₂CO₂—

and amido betaines of the formula:

R⁶—CONH(CH₂)ₜ—N⁺—(R⁷)(R⁸)CH₂CO₂

where t is 2 or 3.

In both formulae R⁶, R⁷ and R⁸ are as defined previously. R⁶ may, in particular, be a mixture of C₁₂ and Ca alkyl groups derived from coconut oil so that at least half, preferably at least three quarters of the groups R⁶ have 10 to 14 carbon atoms. R⁷ and R⁸ are preferably methyl.

Another option is that the zwitterionic surfactant is a sulphobetaine of the formula:

R⁶—N⁺—(R⁷)(R⁸)(CH₂)₃SO₃⁻

or

R⁶—CONH(CH₂)ᵤ—N⁺—(R⁷)(R⁸)(CH₂)₃SO₃⁻

where u is 2 or 3, or variants of these in which —(CH₂)₃SO₃⁻ is replaced by —CH₂C(OH)(H)CH₂SO₃⁻.

In these formulae, R⁶, R⁷ and R⁸ are as previously defined.

Illustrative examples of the zwitterionic surfactants suitable for use include betaines like coco betaine, cocoamidopropyl betaine, lauryl betaine, laurylhydroxy sulfobetaine, lauryldimethyl betaine, cocoamidopropylhydroxylsulfo betaine, behenyl betaine, capryl/capramidopropyl betaine, cocodimethyl carboxymethyl betaine, stearyl betaine. laurylamidopropyl betaine, mixtures thereof or the like. An additional zwitterionic surfactant suitable for use includes lauryl hydroxysultaine and/or cocamidopropyl sultaine. In a preferred embodiment, the zwitterionic surfactant used in the liquid composition of this invention is cocamidopropyl betaine.

Zwitterionic surfactants, when used, may make up from 0.0001 to 15%, and preferably, from 1.0 to 12%, and most preferably, from 1.5 to 10% by weight of the liquid composition. The liquid composition can comprise from 0.0001 to 6.5% by weight zwitterionic surfactant, or 1.5 to 5.5% or from 1.6 to 4% by weight of the liquid composition or no (0.0% by weight) zwitterionic surfactant. In another embodiment of the invention zwitterionic surfactant, like betaine and/or sultaine, when used in the invention, will make up from 0.05 to 50%, and preferably, from 0.08 to 30%, and most preferably, from 0.5 to 20% by weight of the total weight of zwitterionic and anionic surfactant used in the liquid composition. In still another embodiment, betaine and/or sultaine make(s) up from 1 to 12% and anionic surfactant makes up from 2 to 15% by weight of the liquid composition. In still another embodiment, betaine and/or sultaine make(s) up from 1.5 to 7% and anionic surfactant makes up from 2.5 to 8% by weight of the liquid composition. In still another embodiment, when betaine and/or sultaine is/are used in the liquid composition of the present invention, the anionic surfactant comprises less than 25%, and preferably less than 20%, and most preferably, less than 15% by weight isethionate based on total weight of anionic surfactant in the liquid composition. In even another embodiment, when betaine and/or sultaine is/are used in the liquid composition of the present invention, the anionic surfactant comprises more than 15%, and preferably, more than 20%, and most preferably, more than 25% by weight taurate based on total weight of anionic surfactant in the liquid composition. In yet another embodiment, when betaine and or sultaine is/are used, the anionic surfactant is from 0.001 to 7.5% by weight taurate based on total weight of the liquid composition or from 3.5 to 5.5% by weight taurate.

In still another embodiment of the invention, anionic surfactant and zwitterionic surfactant are used at a weight ratio of anionic to zwitterionic surfactant of 5:1 to 1.35:1 or from 4:1 to 3.5:1 or from 3.5:1 to 2.75:1 or from 3:1 to 2.65:1. In even another embodiment of the invention, the anionic surfactant is a taurate, isethionate or both and the zwitterionic surfactant is a betaine. In an often desired embodiment, the anionic surfactant is a taurate and the zwitterionic surfactant is a betaine employed in the liquid composition at the defined weight ratios.

In a further embodiment, when the anionic surfactant used is 95 to 100% by weight taurate (based on total weight of anionic surfactant), 7 to 40%, and preferably, 8 to 35%, and most preferably, 9 to 32% by weight zwitterionic surfactant (like betaine) is used based on total weight of anionic and zwitterionic surfactant used in the liquid composition, whereby when this is the case from 1.8 to 3.2%, or from 2 to 3%, or from 2.25 to 2.75% by weight acid having a pKa from 1 to 8 and a log P from 0 to 4.95 is used based on total weight of the liquid composition and especially when such composition has a pH from 3.5 to 4.5.

Nonionic surfactants may optionally be used in the liquid composition of the present invention. When used, nonionic surfactant typically is included at levels as low as 0.05, 0.15, 1, 1.5 or 2% by weight and at levels as high as 6, 8, 10 or 12% by weight of the liquid composition. The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₃) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

Nonionic surfactants optionally used can include fatty acid/alcohol ethoxylates having the following structures a) HOCH₂(CH₂)ₛ(CH₂CH₂O)ᵥH or b) HOOC(CH₂)_{c}(CH₂CH₂O)_{d}H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{z}H, where i, k are each independently 5 to 15; and z is 5 to 50. In another embodiment of the invention, i and k are each independently 6 to 12; and z is 15 to 35.

The nonionic may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

In an embodiment of the invention, cationic surfactants may optionally be used in the liquid composition of the present invention. One class of optional cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and strearyl amidopropyl dimethylamine lactate.

Even other optional cationic surfactants suitable for optional use include quaternized hydrolysates of silk, wheat, and keratin proteins, and it is within the scope of the invention to use mixtures of the aforementioned cationic surfactants.

If used, cationic surfactants will make up no more than 1.75% by weight of the liquid composition. When present, they typically make up from 0.001 to 0.75%, and more typically, from 0.01 to 0.5% or 0.0% by weight of the liquid composition.

In an embodiment of this invention, the liquid composition of this invention may optionally comprise from 0.0001 to 0.5% by weight polymeric quaternary ammonium compounds (including salts of the same). In another embodiment, the liquid composition will comprise less than 0.3% by weight polymeric quaternary ammonium compounds. In yet another embodiment, the liquid composition comprises less than 0.01% by weight polymeric quaternary ammonium compounds. In even another embodiment, the liquid composition is free of polymeric quaternary ammonium compounds (i.e., 0.0% by weight).

The surfactants suitable for use in the present invention are available from suppliers like Stepan Company, Clariant AG, Croda, Galaxy Surfactants, Sino Lion, Innospec, Dow Chemical and the like.

As to the acid suitable for use in the present invention, the same is limited only to the extent that it is suitable for use in a consumer product, has a pKa from 1 to 8 and a log P from 0 to 4.95. Such an acid is meant to include acids that are mono- and dicarboxylic acids, unsaturated acids, aromatic acids, phosphonic acids or mixtures thereof.

In an embodiment of the invention the acid (or mixture of acids used) has a pKa from 1.5 to 7, and preferably, from 2 to 6.5, and most preferably, from 2 to 5.5. In another embodiment of the invention, the pKa of the acid used is from 2.5 to 4.8. In still another embodiment, the pKa is from 2.75 to 3.25. In one embodiment of the invention, the log P of the acid or acids used is from 0.65 to 4, and preferably, from 1 to 3.3, and most preferably, from 1.2 to 3. In yet another embodiment, the log P of the acid is 1.4 to 2.8 or 1.6 to 2.6.

As to the carboxylic acids suitable for use, illustrative examples include propionic, n-butryic, isobutryic, pentanoic, hexanoic, heptanoic, octanoic, nonanoic and/or decanoic acid. Dicarboxylic acids suitable for use include adipic and/or pimelic acid. The carboxylic acids can also include those which are unsaturated like but-3-enoic, but-2-ynoic, crotonic, isocrotonic, sorbic, angelic, tiglic, and/or tetrolic acid. It is also within the scope of the invention to use or include with other acids those classified as aromatic acids. The aromatic acids suitable for use include coumaric, benzoic, p-amino benzoic, salicylic, capryloyl salicylic, 3-hydroxybenzoic, 4-hydroxybenzoic and/or gallic acid. Phosphonic acids suitable for use include 4-ethoxyphenylphosphonic, 4-methylphenylphosphonic, 4-chlorophenylphosphonic, phenylphosphonic acid or a mixture thereof.

It is within the scope of the invention to include any combination of the acids having a pKa from 1 to 8 and a log P from 0 to 4.75 as described herein. In an embodiment of the invention, the acid used is benzoic, p-aminobenzoic, capryloyl salicylic acid and/or salicylic acid. In another embodiment, the acid used comprises salicylic acid and benzoic acid at a weight ratio of 5:95 to 95:5, and preferably, from 25:75 to 75:25, and most preferably, from 40:60 to 60:40. In another embodiment of the invention, the acid used consists essentially of salicylic acid and benzoic acid at a weight ratio of 5:95 to 95:5, and preferably, from 25:75 to 75:25, and most preferably, from 40:60 to 60:40. In even another embodiment of the invention, the acid used is salicylic acid and benzoic acid at a weight ratio of 5:95 to 95:5, and preferably, from 25:75 to 75:25, and most preferably, from 40:60 to 60:40.

In still another embodiment of the invention, the acid used is all (100% by weight of the acid having a pKa from 1 to 8 and a log P from 0 to 4.75) benzoic acid or all salicylic acid.

The acid used will make up from 0.2 to 8%, and preferably, from 0.5 to 7%, and most preferably, from 1 to 6% or from 1 to 4.5%, or from 1.5 to 3.5% or from 2 to 3% by weight of the liquid composition.

The liquid composition of the invention can be one which is isotropic or lamellar. If lamellar, the same can include from 0.1 to 12%, and preferably, 0.15 to 10%, and most preferably, 0.2 to 8% by weight of a liquid (i.e., lamellar) structuring agent based on total weight of the liquid composition of the invention.

Such structuring agents are typically fatty acids (or ester derivatives thereof) or fatty alcohols. Such materials include C₈-C₂₂ acids such as the following: caprylic acid, lauric acid, myristic acid, oleic acid, isostearic acid, linoleic acid, linolenic acid, ricinoleic acid, elaidic acid, arachidonic acid, myristoleic acid, palmitoleic acid, mixtures thereof or the like. Ester derivatives include propylene glycol isostearate, propylene glycol oleate, glyceryl isostearate, glyceryl oleate and polyglyceryl diisostearate, mixtures thereof or the like. Regarding the fatty alcohols suitable for use, these include cetyl alcohol, stearyl alcohol, behenyl alcohol, mixtures thereof or the like.

The structuring agent is preferably a fatty acid. More preferably, the structurant is selected from the group consisting of caprylic acid, lauric acid, myristic acid or a mixture thereof. In an especially preferred embodiment, the structuring agent is lauric acid.

Water soluble/dispersible polymers are an optional ingredient often desired for inclusion in the liquid composition of the invention in order to aid in enhancing and stabilizing the composition stability and viscosity. The water soluble/or dispersible polymer used can be cationic, anionic, amphoteric or nonionic and have a weight average molecular weight typically higher than 100,000 Daltons. Such polymers make up from 0.05 to 6%, and preferably, 0.07 to 5%, and most preferably, from 0.1 to 4%, by weight of the total weight of the liquid composition.

Examples of such polymers useful in the present invention include the carbohydrate gums such as cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum and mixtures thereof; modified and nonmodified starch granules with gelatinization temperatures between 30 to 85° C. and pregelatinized cold water soluble starch; polyacrylate; Carbopols; alkaline soluble emulsion polymer such as Aculyn 28, Acuyln 22 or Carbopol Aqua SF1; cationic polymer such as modified polysaccharides including cationic guar available from Rhone Poulenc under the trade name Jaguar C13S, Jaguar C14S, Jaguar C17, or Jaguar C16; cationic modified cellulose such as UCARE Polymer JR 30 or JR 40 from Amerchol; N-Hance 3000, N-Hance 3196, N-Hance GPX 215 or N-Hance GPX 196 from Hercules; synthetic cationic polymer such as MerQuat 100, MerQuat 280, Merquat 281 and Merquat 550 by Nalco; cationic starches, e.g., StaLok® 100, 200, 300 and 400 made by Galactasol 800 series by Henkel, Inc.; Quadrosoft Um-200; and Polyquaternium-24.

Gel forming polymers such as modified or non-modified starch granules, xanthan gum, Carbopol, alkaline-soluble emulsion polymers and cationic guar gum such as Jaguar C13S, and cationic modified cellulose such as UCARE Polymer JR 30 or JR 40 are also suitable for use. Citrus peel fibers, such as those made available from suppliers like Cargill under the FiberDesign™ Sensation name, are also suitable for use.

Fatty acid may be included in the liquid composition of the present invention, the same is often a blend of C₈ to C₁₈, preferably C₂ to C₁₈, and most preferably, C₁₆ to C₁₈ fatty acid. Especially preferred is palmitic and/or stearic acid, and most preferably, stearic acid.

Additional fatty acids suitable to optionally include are unsaturated fatty acids like linoleic acid (C18:2), oleic acid (C18:1) or mixtures thereof. In an embodiment of the invention, when fatty acid is used, the same makes up less than 3%, and preferably, less than 2%, and most preferably, less than 1% by weight of the total weight of the liquid composition. In another embodiment, fatty acid makes up from 0.0001 to 0.5% by weight of the total weight of liquid composition where no more than 35%, and preferably, no more than 25%, and most preferably, no more than 18% by weight of the total weight of unsaturated fatty acid used in the bar composition is oleic acid.

Regarding soaps suitable for optional use in the liquid composition of the present invention, the same can include salts that are C₈ to C₁₄ and/or C₁ to C₁₈ soaps including sodium, potassium or ammonium soaps of lauric, palmitic or stearic acid (laurate, palmitate and/or stearate soap).

When optionally used, soap typically makes up from 0.0001 to 6.5%, and preferably, from 0.1 to 5%, and most preferably, from 1.5 to 2.25% by weight of the liquid composition. Liquid composition with 0.0% by weight soap is also within the scope of the invention.

Water typically makes up from 35 to 85%, and preferably, from 45 to 80%, and most preferably, from 55 to 80% by weight of the liquid composition prior to dilution with water to yield aqueous or wash solution.

As to the wash solution, the pH of the same is from 2.9 to 5.2, and preferably, from 3.3 to 4.9, and most preferably, from 3.9 to 4.8. In another embodiment, the pH of the wash solution is from 4 to 4.7 or from 4.3 to 4.6. The liquid composition (i.e., the end use composition) solution has viscosity from 2,000 to 15,000 mPa·s, preferably, from 3,000 to 10,000 mPa·s, and most preferably, from 4,000 to 9,000 mPa·s. The wash solution will have a viscosity from 20 to 1,500 mPa·s, and preferably, from 30 to 1,250 mPa·s, and most preferably, from 40 to 1,000 mPa·s.

Optional but preferred ingredients that may be used in the liquid composition of the present invention include preservatives to assist against the growth of potentially harmful microorganisms when the end use wash composition is made. Suitable traditional preservatives that may be used include propionate salts, and a variety of quaternary ammonium compounds. Often preferred preservatives are potassium sorbate, iodopropynyl butyl carbamate, phenoxyethanol, wasabi-based preservatives, imidazolidinyl urea, sodium dehydroacetate and benzyl alcohol. Often desired additives suitable to be employed in the liquid composition of the present invention are 1,2-alkanediols like 1,2-octanediol, 1,2 hexanediol or mixtures thereof.

The liquid composition may comprise of a preservative system that is formaldehyde-free, paraben-free, or both.

When employed, the traditional preservatives, and/or vicinal diol component will not make up collectively more than 2%, and preferably, not more than 1%, and most preferably, from 0.2 to 0.85% by weight of the liquid composition. In an embodiment of this invention, from 0.2 to 0.8% by weight preservative, and/or vicinal diol component is used, based on total weight of the liquid composition. Preferably, preservative is used in the liquid composition of this invention.

Traditional fragrance components like eugenol, coumarin, linalyl acetate, citronellal, iris concentrate, terpinyl acetate, terpineol, thymol, pinenes (e.g., alpha and beta pinene) and citronellol may optionally be added to the liquid composition. Such components make up from 0.01 to 1.2%, and preferably, from 0.1 to 1%, and most preferably, from 0.1 to 0.65% by weight of the liquid composition when used.

Other optional ingredients suitable for use include zinc pyrithione, octopirox, or a mixture thereof, especially when the liquid composition is used as a shampoo that provides antidandruff benefits. Each of these substances may range from 0.05 to 3%, and preferably, between 0.1 and 2% by weight of the total weight of the liquid composition.

Additional optional ingredients that may be used include sensory oils and/or exfoliants. Desirable oils include rose, lime, coconut, lavender, argan, sweet almond oil(s) or mixtures thereof. Illustrative exfoliants desirable for use include salt, sugar, apricot, walnut shell, rice, nutmeg and/or oatmeal powder(s). When used, sensory oils and exfoliants can make up from 0.1 to 2% by weight of liquid composition, with the proviso that the total amount of fragrance and sensory oil does not exceed 2.5% by weight of the composition, and preferably, not more than 2.0 percent by weight of the composition.

The liquid composition may include vitamins. Illustrative vitamins include Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of optional vitamins when present may range from 0.001 to 5%, and preferably from 0.01% to 3%, optimally from 0.1 to 1% by weight of the composition.

Other optional additives suitable for use include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, thiamidol; alpha- and/or beta-hydroxyacids; retinoic acid and its derivatives (e.g., cis and trans); retinal; retinol; retinyl esters such as retinyl acetate, retinyl palmitate, and retinyl propionate; petroselinic acid; conjugated linoleic acid; 12-hydroxystearic acid; mixtures thereof or the like. Conditioning agents such as polyquaternium compounds (e.g., polyquaternium-67) may also be desirable for inclusion in the inventive composition. Such additives, when used, collectively make up from 0.001 to 3%, preferably, from 0.01 to 2%, and, most preferably, from 0.1 to 1.5% by weight of the liquid composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Amounts of these materials when present may range from 0.01 to 3%, and, preferably, from 0.1 to 2% by weight of the liquid composition.

A variety of herbal extracts may optionally be included in the cosmetic wash compositions of this invention. Illustrative extracts include those removed from green tea, yarrow, ginseng, marigold, hibiscus, ginko biloba, chamomile, licorice, aloe vera, grape seed, citrus unshiu, willow bark, sage and rosemary. Humectants like glycerol and other polyols (e.g., sorbitol) may also be included. Humectants and/or extracts, such as sorbitol, when used, typically make up from 0.01 to 5%, preferably, from 0.01 to 4%, and most preferably, from 0.02 to 3% by weight of the liquid composition.

Another optional additive suitable for use includes hemp oil with 2.5 to 25% by weight cannabigerol and/or cannabidiol at from 0.5 to 10 percent by weight. When used, such oil makes up from 0.0001 to 5% by weight of the composition, and preferably, from 0.01 to 3% by weight of the liquid composition.

Also, optionally suitable for use include materials like chelators (e.g., EDTA), opacifiers (like TiO₂, particle size from 50 to 1200 nm, and preferably, 50 to 350 nm), kaolin, bentonite, zinc oxide, iron oxide, mica, C₈₋₂₂ fatty acid substituted saccharides, lipoic acid, retinoxytrimethylsilane (available from Clariant Corp. under the Silcare 1M-75 trademark), dehydroepiandrosterone (DHEA) or mixtures thereof. Ceramides (including Ceramide 1, Ceramide 3, Ceramide 3B and Ceramide 6) as well as pseudoceramides may also be optionally included as can 10-hydroxystearic acid. Amounts of these additional and optional materials, when used, may range from 0.0001 to 3%, and preferably, from 0.001 to 2%, and most preferably, from 0.001 to 1.5% by weight of the liquid composition.

Colorants or dyes may also be included in the bar compositions. These substances may range from 0.05 to 5%, and preferably, between 0.1 to 2% by weight of the liquid composition.

Conditioning agents like hydroxypropyltrimonium chloride, 5-ureidohydantoin and/or glyoxyldiureide may be used. The components when used make up from 0.5 to 4%, and, preferably, from 0.75 to 3%, and most preferably, from 1 to 2% by weight of the liquid composition.

Sunscreen actives may also be optionally included in the bar composition of the present invention. Particularly preferred are such materials as ethylhexyl p-methoxycinnamate, available as Parsol MCX®, Avobenzene, available as Parsol 1789© and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.01 to 3%, and preferably, from 0.5 to 2%, optimally, from 0.75 to 1.5% by weight of the liquid composition.

Conventional buffers/pH modifiers may be used. These include commonly employed additives like sodium hydroxide, potassium hydroxide, hydrochloric acid, citric acid/citrate buffers, triethanolamine, or mixtures thereof where pH is determined using a Thermo Fisher Scientific pH meter.

Optional antimicrobial ingredients may be used but are not required. Illustrative ingredients include quaternary ammonium compounds like cetylpyridinium chloride, cetyltrimethylammonium chloride, cetyltrimethylammonium bromide, benzalkonium chloride, benzethonium chloride, cetrimide or mixtures thereof. Cetrimonium chloride and/or bromide, chloroxylenol, antimicrobial lipids, antimicrobial peptides or mixtures thereof may also be used. Triclosan may also be used but is not generally desired. Mixtures of thymol and terpineol are often desired for use, typically at a weight ratio from 1:3 to 3:1 or from 1:2 or 2:1 or from 1:0.7 to 0.7:1. When used, such optional antimicrobial ingredients make up from 0.001 to 6%, and preferably, from 0.01 to 5%, and most preferably, from 0.01 to 2.5% by weight of the liquid composition.

When preparing liquid composition conventional techniques may be used, and these include moderate shear mixing at atmospheric pressure and temperatures between 25 to 50° C.

A wide variety of packaging can be employed with the liquid composition of this invention. Laminated boxes and bags as well as plastic containers may be used. Preferably, the packaging used with the composition of the present invention is packaging made from recycled material like post-consumer resins or biodegradable material. Most preferably, the liquid composition is sold in biodegradable packaging material, like post-consumer resins.

The following examples are provided to facilitate an understanding of the present invention. The examples are not intended to limit the scope of the claims.

### Example I

The formulations depicted in Table I as Samples 1 A-D were made consistent with the present invention. The formulation of Sample 1 E is provided as a comparative formulation. All weight percents (Weight %) are based on total weight of active in the liquid composition made.

TABLE I

| | 1A | 1B | 1C | 1D | 1E |
|---|---|---|---|---|---|
| | Weight | Weight | Weight | Weight | Weight |
| Ingredient | % | % | % | % | % |
| Sodium Methyl | 4.2 | 4.2 | 4.2 | 5.4 | 5.4 |
| Lauroyl Taurate | | | | | |
| Cocoamidopropyl | 1.8 | 1.8 | 1.8 | 0.6 | 0.6 |
| betaine | | | | | |
| Sodium Benzoate* | 2.5 | 2.5 | 2.5 | 2.4 | 0.4 |
| Caprylic acid | 0 | 0 | 0 | 1.0 | 0 |
| Cocoamide | 0 | 0.5 | 0.5 | 0.5 | 0.5 |
| monoethanolamide | | | | | |
| Fatty Acid | 0.75 | 0.56 | 0.56 | 0.31 | 0.31 |
| Glycerol | 0.1 | 0 | 0 | 0 | 0 |
| monostearate | | | | | |
| Polymeric | 4.3 | 4.3 | 4.75 | 0 | 0 |
| Rheology Modifier | | | | | |
| pH Adjuster | As | As | As | As | As |
| | Needed | Needed | Needed | Needed | Needed |
| Fragrance | — | — | 0.5 | 0.5 | 0.5 |
| Glycerin | 5 | 5 | 5 | 5 | 5 |
| Opacifier | 0.34 | 0.34 | 0.34 | 0 | 0 |
| Water | qs 100 | qs 100 | qs 100 | qs 100 | qs 100 |
| Formulation pH | 4 | 4 | 4 | 4 | 4 |

*Benzoic acid at liquid composition pH

### Example II

### Method for Antimicrobial Efficacy Evaluation

The antimicrobial efficacy of Formulation Samples in Table I were evaluated via a procedure similar to the one described in ASTM International testing method ASTM E2315-16, entitled “Standard Guide for Assessment of Antimicrobial Activity Using a Time-Kill Procedure,” incorporated herein by reference. The data is provided in Table II.

E. coli ATCC 10536 and S. aureus ATCC 6538 were used in the study to represent Gram negative and Gram positive bacteria, respectively. The bacteria were stored at −80° C. Fresh isolates were cultured twice on Tryptic Soy Agar plates for 24 hours at 37° C. before each experiment. Growth-phase bacterial cultures at 1.5×10⁸ to 5×10⁸ colony forming units per mL (CFU/mL) were prepared from these cultures and used as the challenge innoculum for the experiment. Dilutions of 50% test Formulations were exposed to the innoculum at 25° C. After 10, 20, 30, or 60 seconds of exposure, the mixtures were neutralized to arrest the antimicrobial activity of the test solutions. The neutralized bacteria solutions were serially diluted, plated on solid medium, incubated for 24 hours, and viable cells were enumerated through colony counting. Bactericidal activity is defined as the log reduction in CFU/mL relative to cultures not exposed to any test solutions i.e., no-treatment control. The log reduction was calculated using the formula: Log₁₀ Reduction=Log₁₀ (no-treatment control CFU)−Log₁₀(test sample CFU).

TABLE II

| Efficacy Test vs Organism | | 1B | 1C | 1D | 1E |
|---|---|---|---|---|---|
| S. | Log reduction @ | 0.9 | 1.22 | >3.9 | 0.10 |
| Aureus | 10 sec | | | | |
| | Log reduction @ | 2.6 | 2.70 | >3.9 | 0.31 |
| | 20 sec | | | | |
| | Log reduction @ | >2.8 | >2.8 | >3.9 | 0.46 |
| | 30 sec | | | | |
| | Log reduction @ | >2.8 | >2.8 | >3.9 | 1.26 |
| | 60 sec | | | | |
| E. | Log reduction @ | >3.0 | >3.0 | >4.0 | 0.77 |
| coli | 10 sec | | | | |
| | Log reduction @ | >3.0 | >3.0 | >4.0 | 3.39 |
| | 20 sec | | | | |
| | Log reduction @ | >3.0 | >3.0 | >4.0 | >4.19 |
| | 30 sec | | | | |
| | Log reduction @ | >3.0 | >3.0 | >4.0 | >4.19 |
| | 30 sec | | | | |

The data in Table II shows that liquid compositions made consistent with the present invention, 1B, 1C and 1D unexpectedly display excellent antimicrobial efficacy against both Gram positive and Gram negative organisms, providing at least a 1 log reduction after 10 seconds of contact, a 2 log reduction after 20 seconds of contact or a 3 log reduction after 30 seconds of contact. By contrast, the comparative liquid composition, 1 E, displayed marginal efficacy, especially against Gram positive bacteria when the level of organic acid was reduced.

Furthermore, trained panelists repeated hand washes with the liquid compositions consistent with the present invention (1A-1D) as well as compositions with reduced levels of organic acid (1 E). Unexpectedly, all panelists concluded the liquid compositions made according to the invention and with higher levels or organic acid were at least as mild on skin as the comparative formula with less organic acid.

### Example III

### Mildness Assessment

The liquid compositions made consistent with the present invention were evaluated for skin mildness using a Zein solubilization assay.

Zein powder, 0.3 grams, (Sigma. 23625-1 kg, lot #SLBZ2926 or Acros Organic™ Zein) was added to 10 ml of a 5% dilution of the test formulations and mixed for 30 mins at room temperature. After 30 minutes, the resulting samples were visually inspected to determine if excess undissolved zein was present. In cases where excess zein was not present, an additional 0.3 grams of Zein was added and mixed for an additional 30 mins. The resulting sample was then filtered using a 0.45 micron filter. The filtrate was diluted in a 2% sodium dodecyl sulfate solution and the absorbance at 298 nm was measured to determine the amount of dissolved Zein. A control experiment with liquid composition deplete of Zein was prepared in a similar way and measured at 298 nm to measure background. The amount of dissolved Zein is reported as:

Zein Absorbance=[Absorbance (298 nm) of Zein of liquid composition−Absorbance (298 nm) of background sample]*dilution factor. Milder products dissolve less Zein and give lower absorbance values. The range of Zein absorbance values that enable a ranking of mildness of products in terms of ability to irritate skin are defined and described in Table Ill below.

TABLE III
Zein absorbance values for liquid compositions

| | Zein Absorbance Values |
|---|---|
| Mild/minimal or no skin irritation | ≤7 |
| Moderately harsh/some irritation | 8-19 |
| Harsh/high skin irritation | ≥20 |

Zein absorbance values for all compositions consistent with the present invention and as shown in Table I (1A-1D) were measured to be 3 to 7, unexpectedly indicating that such compositions are mild on skin and yet have excellent antimicrobial efficacy.

### Example IV

### Comparative Samples of Liquid Compositions Having Different Organic Acid Types

antimicrobial efficacy for S. aureus at 10 seconds and using the test procedure described in Example II was repeated for compositions with varying organic acids as shown in Table IV. The data indicates that liquid compositions consistent with the present invention (4A, 4B, 4C and 4D); and therefore, having acid of the claimed criteria, unexpectedly displayed excellent antimicrobial efficacy. In contrast, liquid compositions with organic component outside (pKa=15.4, log P=1.1) the specified claim criteria (4E), have marginal to no efficacy.

TABLE IV

| | 4A | 4B | 4C | 4D | 4E |
|---|---|---|---|---|---|
| | Weight | Weight | Weight | Weight | Weight |
| Ingredient | % | % | % | % | % |
| Sodium Methyl | 4.2 | 4.2 | 4.2 | 4.2 | 4.2 |
| Lauroyl Taurate | | | | | |
| Cocoamidopropyl | 1.8 | 1.8 | 1.8 | 1.8 | 1.8 |
| betaine | | | | | |
| Sodium Benzoate | 2.5 | 0 | 0 | 0 | 0 |
| (pKa = 4.2; log P = | | | | | |
| 1.8) | | | | | |
| Caprylic acid (pKa = | 0 | 2.05 | 0 | 0 | 0 |
| 4.72, log P = 3.05) | | | | | |
| Hydrocinnamic acid | 0 | 0 | 2.05 | 0 | 0 |
| (pKa = 4.37, log P = | | | | | |
| 1.8) | | | | | |
| Capryloyl salicylic | 0 | 0 | 0 | 2.06 | 0 |
| acid (pKa = 3.83, log | | | | | |
| P = 4.83) | | | | | |
| Benzyl alcohol (pKa = | 0 | 0 | 0 | 0 | 2.06 |
| 15.4, log P = 1.1) | | | | | |
| Cocoamide | 0.5 | 0.5 | 0.5 | 0.5 | 0.5 |
| monoethanolamide | | | | | |
| Fatty Acid | 0.31 | 0.31 | 0.31 | 0.31 | 0.31 |
| Structuring Agent | | | | | |
| NaOH to adjust pH | <1 | <1 | <1 | <1 | <1 |
| Fragrance | 0.5 | 0.5 | 0.5 | 0.5 | 0.5 |
| Glycerin | 5 | 5 | 5 | 5 | 5 |
| Water | qs 100 | qs 100 | qs 100 | qs 100 | qs 100 |
| pH | 4 | 4 | 4 | 4 | 4 |
| Log Reduction S. | 1.07 | 1.33 | 2.89 | >3.52 | 0.34 |
| Aureus @ 10 sec | | | | | |

### Example V

antimicrobial efficacy for S. aureus @ 10 sec using testing procedure as described above for formulations with an organic acid is shown in Table V to demonstrate the unexpected additional synergistic effect of the organic acid and surfactant. The data surprisingly shows antimicrobial efficacy is enhanced when both anionic surfactant and preferred levels of the organic acid are used. In contrast the surfactant with low level of the organic acid (5) or the organic acid alone without the anionic surfactant (50) provide no antimicrobial efficacy. All weight percents (Weight %) are based on total weight of active in the liquid composition made.

TABLE V

| | | 5A | 5B | 5C |
|---|---|---|---|---|
| Ingredient | | Weight | Weight | Weight |
| classification | Ingredient | % | % | % |
| Anionic surfactant | Sodium Methyl | 4.2 | 4.2 | 0 |
| | Lauroyl Taurate | | | |
| Amphoteric or | Cocoamidopropyl | 1.8 | 1.8 | 0 |
| zwitterionic | betaine | | | |
| cosurfactants | | | | |
| Organic acid | Sodium Benzoate | 3.02 | 0.4 | 2.97 |
| Foam Booster | Cocoamide | 0.5 | 0.5 | 0.5 |
| | monoethanolamide | | | |
| Structuring agents | Stearic acid | 0.31 | 0.31 | 0.31 |
| pH adjusters | Sodium hydroxide, | <1 | <1 | <1 |
| | hydrochloric acid or | | | |
| | citric acid as needed | | | |
| Fragrance | Fragrance | 0.5 | 0.5 | 0.5 |
| Humectants | Glycerin | 5 | 5 | 5 |
| Water | Water | qs 100 | qs 100 | qs 100 |
| pH | | 4 | 4 | 4 |

| | | | |
|---|---|---|---|
| Log Reduction S. Aureus @ 10 sec | 1.97 | −0.02 | −0.07 |

### Example VI

Antimicrobial efficacy for S. aureus @ 10 seconds was assessed using the testing procedure described above in this Example for liquid compositions with a varying ratio of anionic to zwitterionic surfactant with the same organic acid. The data in Table VI unexpectedly shows that superior efficacy is achieved when the anionic to zwitterionic ratio is increased.

TABLE VI

| | 6A | 6B | 6C |
|---|---|---|---|
| Ingredient | Weight % | Weight % | Weight % |
| Sodium Methyl Lauroyl | 4.2 | 3 | 1.8 |
| Taurate | | | |
| Cocoamidopropyl | 1.8 | 3 | 4.2 |
| betaine | | | |
| Sodium Benzoate | 3.02 | 3.03 | 2.97 |
| Cocoamide | 0.5 | 0.5 | 0.5 |
| monoethanolamide | | | |
| Stearic acid | 0.31 | 0.31 | 0.31 |
| Sodium hydroxide, | <1 | <1 | <1 |
| hydrochloric acid or | | | |
| citric acid as needed | | | |
| Fragrance | 0.5 | 0.5 | 0.5 |
| Glycerin | 5 | 5 | 5 |
| Water | qs 100 | qs 100 | qs 100 |
| pH | 4 | 4 | 4 |
| Log Reduction | 1.97 | 0.51 | 0.07 |
| S. Aureus @ 10 sec | | | |

1. A liquid composition comprising:
a) from 2 to 20% by weight anionic surfactant;
b) from 0.2 to 8% by weight of at least one acid having a pKa from 1 to 8 and a log P from 0 to 4.95, wherein the acid is benzoic acid, salicylic acid or a mixture thereof;
c) water; and
d) optionally, a liquid structuring agent,
wherein the composition in a 50% aqueous solution has a pH from 2.9 to 5.2 and further wherein the composition is substantially sulfate free, wherein the anionic surfactant is 95 to 100% by weight of a taurate based on total weight of anionic surfactant, and the composition further comprises 7 to 40%, by weight zwitterionic surfactant based on total weight of anionic and zwitterionic surfactant used in the liquid composition, whereby 1.8 to 3.2%, by weight acid having a pKa from 1 to 8 and a log P from 0 to 4.95 is used based on total weight of the liquid composition when the composition has a pH from 3.5 to 4.5.

2. The liquid composition according to claim 1 wherein the anionic surfactant is a taurate, isethionate or a mixture thereof, the composition comprising no more than 3.5% by weight sulfate, is isotropic or lamellar and is a shampoo, wash for the hands, body and/or face, or is a home care cleanser.

3. The liquid composition according to claim 1 wherein the acid is propionic, n-butryic, isobutryic, pentanoic, hexanoic, heptanoic, octanoic, nonanoic and/or decanoic acid, adipic, but-3-enoic, but-2-ynoic, crotonic, isocrotonic, sorbic, angelic, tiglic, tetrolic, coumaric, benzoic, p-amino benzoic, salicylic, capryloyl salicylic, 3-hydroxybenzoic, 4-hydroxybenzoic, gallic, ethoxyphenylphosphonic, 4-methylphenylphosphonic, 4-chlorophenylphosphonic, phenylphosphonic acid or a mixture thereof.

4. The liquid composition according to claim 1 wherein the acid makes up from 0.5 to 7% by weight of the liquid composition.

5. The liquid composition according to claim 1 wherein the composition has a Zein value of less than or equal to 7.

6. The liquid composition according to claim 1 wherein the composition has an antimicrobial activity in a 50% wash solution that provides a Log₁₀ Reduction against E. coli ATCC 10536 and S. aureus ATCC 6538 of at least 1.0 after 15 seconds of contact with a surface.

7. The liquid composition according to claim 6, wherein the surface is skin, hair, or an inanimate object in need of antimicrobial reduction.

8. The liquid composition according to claim 1 wherein the composition further comprises a zwitterionic surfactant which is a betaine, sultaine or a mixture thereof.

9. The liquid composition according to claim 1 wherein the composition further comprises niacinamide, 12-hydroxystearic acid, a retinol, a resorcinol, thymol, terpineol, benzalkonium chloride or a mixture thereof.

10. A method for reducing both Gram negative and Gram positive antimicrobial presence on a surface in need of antimicrobial reduction comprising the steps of:
a) contacting the surface with a wash solution comprising the liquid composition of any one of claims 1 to 9 and water; and
b) rinsing the wash solution from the surface,
wherein the liquid composition has an antimicrobial activity in a 50% wash solution that provides a Log₁₀ Reduction against E. coli ATCC 10536 and S. aureus ATCC 6538 of at least 1.0 after 15 seconds of contact with a surface that is skin or hair or an inanimate object and further wherein the liquid composition has a Zein value of less than or equal to 7.

11. The method according to claim 10 wherein the liquid composition has an antimicrobial activity in a 50% wash solution that provides a Log₁₀ Reduction against E. coli ATCC 10536 and S. aureus ATCC 6538 of at least 1.0 after after 10 seconds of contact with the surface and further wherein the liquid composition comprises Vitamin B6, Vitamin C, Vitamin E, Folic Acid, Biotin, panthenol, zinc pyrithione, octopirox, retinyl acetate, retinyl palmitate, and retinyl propionate; petroselinic acid, conjugated linoleic acid or a mixture thereof.

12. The method according to claim 10 wherein the liquid composition further comprises cetylpyridinium chloride, cetyltrimethylammonium chloride, cetyltrimethylammonium bromide, benzalkonium chloride, benzethonium chloride, cetrimide or a mixture thereof.

13. The method according to claim 10 wherein the liquid composition further comprises eugenol, coumarin, linalyl acetate, citronellal, iris concentrate, terpinyl acetate, terpineol, thymol, alpha pinene, beta pinene or a mixture thereof.
